(19) United States
(12) Patent Application Publication
(10) Pub. No.: US 2009/0054785 A1
(43) Pub. Date: Feb. 26, 2009

(54) METHOD FOR DETECTING BODY TEMPERATURE OF LIVE BODY

(76) Inventors: Vincent WENG, HsinChu (TW); Kevin Lin, HsinChu (TW)

Correspondence Address: SINORICA, LLC, 528 FALLSGROVE DRIVE, ROCKVILLE, MD, 20850, US

(21) Appl. No.: 12/189,986

(22) Filed: Aug. 12, 2008

(30) Foreign Application Priority Data
Aug. 21, 2007 (TW) 096130830

(51) Int. Cl. A61B 5/01; G01J 5/00

(52) U.S. Cl. 600/474; 374/121

(57) ABSTRACT

The present invention discloses a method for detecting body temperature of a live body. Firstly, an infrared thermometer is used to scan a testee to obtain a plurality of infrared signals. Next, the infrared signals are sequenced to obtain the greatest one thereof. Next, several infrared signals closest to the greatest infrared signal are selected from the sequenced infrared signals. Next, an average infrared signal is worked out from the selected infrared signals. Then, the average infrared signal is converted into a temperature. Thereby, not only the test result is more precise, but also the testee can learn the test result sooner.

### BACKGROUND OF THE INVENTION

1. Field of the Invention

The present invention relates to a method for detecting body temperature of a live body, particularly to a method for fast detecting body temperature of a live body.

2. Description of the Related Art

Temperature is an important physiological index indicating whether a living body is healthy. The instruments for temperature measurement can be categorized into the contact thermometers and the non-contact thermometers. The contact thermometers include mercury thermometers and electronic thermometers. The non-contact thermometers include infrared thermometers, such as infrared ear thermometers, infrared forehead thermometers, and infrared ear-forehead thermometers. The infrared thermometers are getting more and more popular because they are easy to operate and prompt to output results.

The infrared ear thermometer is not always suitable to the persons having some ear defect or sleeping children. Contrarily, the infrared forehead thermometer can work in the abovementioned cases. A U.S. Pat. No. 6,292,685 disclosed an infrared forehead thermometer, wherein the probe of the infrared thermometer is used to scan the testee's forehead to obtain a plurality of measurement values, and the greatest one of the measurement values is converted into a forehead temperature. However, the greatest measurement value usually contains noise. Thus, the test result is likely inaccurate. Besides, the conventional infrared forehead thermometer has to convert each measured infrared signal into a forehead temperature immediately. Thus, the scanning speed thereof is reduced, and the accuracy thereof is decreased.

Accordingly, the present invention proposes another method for detecting body temperature of a live body to overcome the abovementioned problems.

### SUMMARY OF THE INVENTION

One objective of the present invention is to provide a method for detecting body temperature of a live body, which firstly obtains an average infrared signal from several infrared signals and converts the average infrared signal into a forehead temperature, and which outperforms the conventional technology that has to one by one convert infrared signals into temperature values and is hard to fast detect body temperature.

Another objective of the present invention is to provide a method for detecting body temperature of a live body, which can fast obtain a high-accuracy result in temperature measurement.

The method for detecting body temperature of a live body of the present invention comprises steps: scanning the forehead of a testee and receiving a plurality of infrared signals; sequencing the infrared signals to obtain the greatest infrared signal; selecting several infrared signals closest to the greatest infrared signal from the sequenced infrared signals; obtaining an average infrared signal of the selected infrared signals; and converting the average infrared signal into a forehead temperature.

Below, the technical contents of the present invention will be described in detail to enable the persons skilled in the art to easily understand the present invention.

It to be noted that the foregoing general description and the following detailed description are only intended to exemplify the present invention but not to limit the scope of the present invention.

### BRIEF DESCRIPTION OF THE DRAWINGS

FIG. 1 is a perspective view schematically showing an infrared forehead thermometer used in the description of the present invention;

FIG. 2 is a block diagram schematically showing the circuit of an infrared forehead thermometer used in the description of the present invention;

FIG. 3 is a flowchart of a method for detecting body temperature of a live body according to the present invention; and

FIG. 4 is a diagram schematically showing the sequencing of infrared signals according to the present invention.

### DETAILED DESCRIPTION OF THE INVENTION

The present invention proposes a method for detecting body temperature of a live body, which uses an infrared thermometer to fast detect the forehead temperature of a testee and obtain a more precise forehead temperature. Below, an infrared forehead thermometer is used as a measurement instrument to describe the technical contents of the present invention.

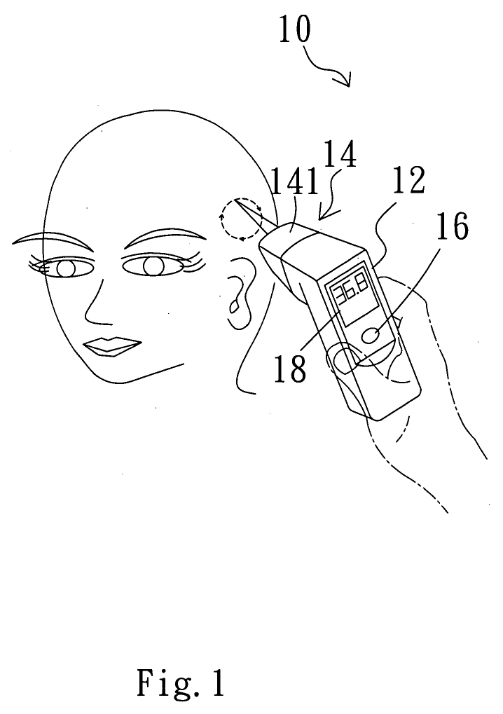

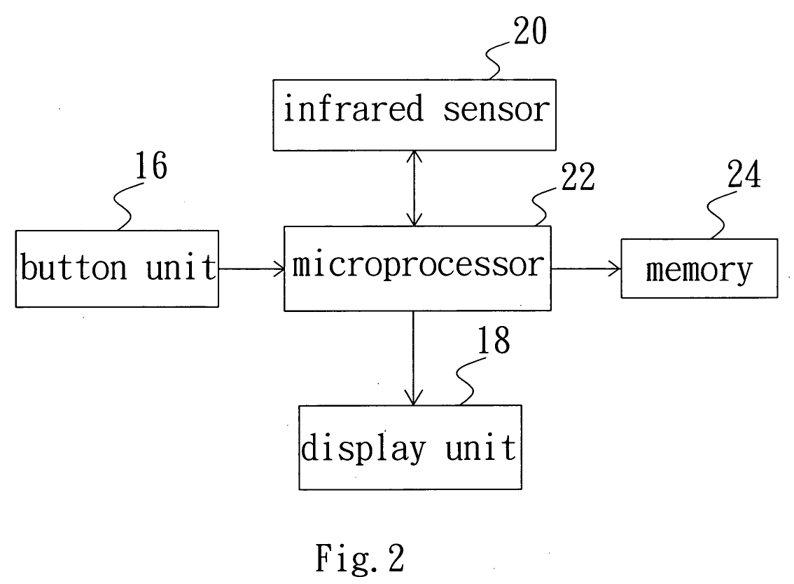

Refer to FIG. 1 and FIG. 2. FIG. 1 is a perspective view schematically showing an infrared forehead thermometer used in the description of the method of the present invention, and FIG. 2 is a block diagram schematically showing the circuit of the infrared forehead thermometer. The infrared forehead thermometer 10 comprises a body 12 and a probe structure 14 at one end of the body 12. The body 12 has a button unit 16 for operation and a display unit 18 presenting the results of measurement, such as a liquid crystal display. The probe structure 14 has a casing 141, an infrared-permeable window at the front end of the casing 141, and an infrared sensor 20 inside the casing 141. The infrared sensor 20 can detect the infrared radiation passing through the window. The infrared forehead thermometer 10 also comprises a microprocessor 22 and a memory 24 both arranged inside the body 12. The microprocessor 22 is coupled to the button unit 16, the display unit 18, the infrared sensor 20 and the memory 24 and controls the operation of the abovementioned components. The measurement results and other data are stored in the memory 24.

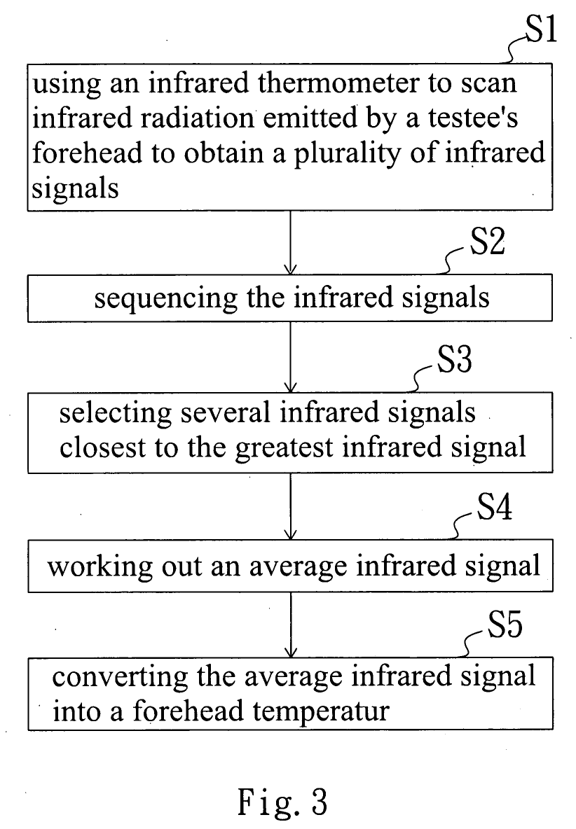

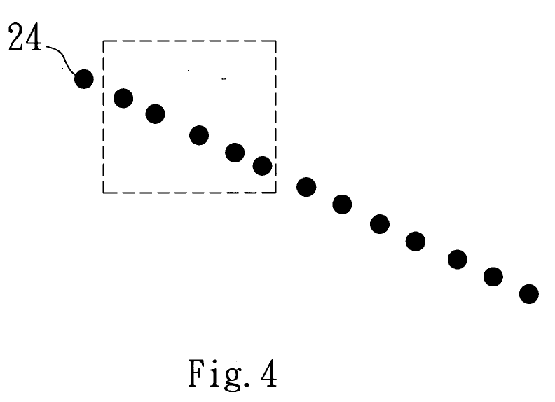

Refer to FIG. 3 a flowchart of a method for detecting body temperature of a live body according to the present invention. In Step S1, the probe structure 14 of the infrared thermometer 10 is aimed at the forehead of a testee and then gradually moved so that the infrared sensor 20 can scan and detect the infrared radiation emitted by the forehead of the testee and then obtain a plurality of infrared signals. Next, in Step S2, the infrared sensor 20 transmits the plurality of infrared signals to the microprocessor 22, and the microprocessor 22 sequences the infrared signals from small to great or from great to small and obtains the greatest infrared signal 24, as shown in FIG. 4. Next, in Step S3, the microprocessor 22 selects several infrared signals closest to the greatest infrared signal 24. Next, in Step S4, the microprocessor 22 works out an average infrared signal from the selected infrared signals. Next, in Step S5, the microprocessor 22 converts the average infrared signal into a forehead temperature. Then, the microprocessor 22 transmits the forehead temperature to the display unit 18, and the user can learn the measurement result from the display unit 18. The infrared thermometer 10 should detect the temperature related to the primary artery in the head. As shown in FIG. 1, the infrared thermometer 10 is scanning the temple area because the artery in the temple interconnects with the heart via the carotid artery.

In conclusion, the method for detecting body temperature of a live body of the present invention sequences the received infrared signals, selects several appropriate infrared signals from the sequenced infrared signals, works out an average infrared signal from the selected infrared signals and converts the average infrared signal into a forehead temperature. The present invention can solve the problem of the conventional technology that has to convert all infrared signals into temperatures one by one and is hard to fast obtain the measurement results. The conventional technology adopts the highest temperature as the measurement output. The highest temperature is worked out from the greatest infrared signal. However, the greatest infrared signal contains a notable proportion of noise. The present invention does not adopt the greatest infrared signal but utilizes several infrared signals closest to the greatest infrared signal. Therefore, the present invention has a more precise measurement result.

The embodiments described above are to demonstrate the technical contents and characteristics of the present invention and to enable the persons skilled in the art to understand, make, and use the present invention. However, it is not intended to limit the scope of the present invention. Therefore, any equivalent modification or variation according to the spirit of the present invention is to be also included within the scope of the present invention.

What is claimed is:

1. A method for detecting body temperature of a live body comprising steps:
using an infrared thermometer to scan infrared radiation emitted by a testee's forehead to obtain a plurality of infrared signals;
sequencing said infrared signals to obtain a greatest infrared signal of said infrared signals, selecting several said infrared signals closest to said greatest infrared signal from said infrared signals sequenced, and working out an average infrared signal from said infrared signals selected; and
converting said average infrared signal into a forehead temperature.

2. The method for detecting body temperature of a live body according to claim 1, wherein said infrared thermometer has an infrared sensor detecting infrared radiation emitted by a testee's forehead.

3. The method for detecting body temperature of a live body according to claim 2, wherein said infrared thermometer has a microprocessor; said microprocessor is coupled to said infrared sensor, receives said infrared signals from said infrared sensor, sequences said infrared signals, works out said average infrared signal, and converts said average infrared signal into a forehead temperature.

4. The method for detecting body temperature of a live body according to claim 2, wherein said infrared thermometer has a probe structure; said probe structure has a casing accommodating said infrared sensor; one front end of said casing has a window, and said infrared sensor receives infrared radiation passing through said window.
